(19) United States
(12) Patent Application Publication
(10) Pub. No.: US 2026/0091245 A1
(43) Pub. Date: Apr. 2, 2026

(54) ENERGY TREATMENT PLAN OPTIMIZATION METHOD AND APPARATUS

(71) Applicant: Siemens Healthineers International AG, Steinhausen (CH)

(72) Inventors: Martin Sabel, Hagendorn (CH); Jarkko Peltola, Tuusula (FI); Esa Kuusela, Espoo (FI); Juha Kauppinen, Espoo (FI)

(21) Appl. No.: 18/898,892

(22) Filed: Sep. 27, 2024

(51) Int. Cl. A61N 5/10

(52) U.S. Cl. CPC A61N 5/1031; A61N 5/1064; A61N 5/1081; A61N 2005/1089

(57) ABSTRACT

For use with a radiation treatment platform having a set of at least two selectable therapeutic energy settings and wherein the radiation treatment platform includes a therapeutic energy source that travels along an arcuate pathway about a given patient during a radiation treatment session (such as, for example, a volumetric modulated arc therapy radiation treatment session), a control circuit can optimize therapeutic energy settings from amongst the set of at least two selectable therapeutic energy settings for different portions of the arcuate pathway to provide optimized therapeutic energy settings and then generate an optimized energy treatment plan as a function, at least in part, of the optimized therapeutic energy settings.

### TECHNICAL FIELD

These teachings relate generally to treating a patient's planning target volume with energy pursuant to an energy-based treatment plan and more particularly to optimizing an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

A radiation treatment plan typically comprises specified values for each of a variety of treatment-platform parameters during each of a plurality of sequential fields. Treatment plans for radiation treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

Many energy-based treatment systems offer a range of discrete photon energies for irradiation. In some cases, in the treatment planning process a particular energy level is manually selected by the treatment planner for each treatment field. Using a typical approach, the plan will use a single energy that will fix the depth dose behavior for the corresponding field. Current approaches in these regards do not necessarily meet all the needs and/or sufficiently leverage the capabilities that characterize all application settings.

### BRIEF DESCRIPTION OF DRAWINGS

The above needs are at least partially met through provision of the energy treatment plan optimization method and apparatus described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings;

FIG. 3 comprises a graph as configured in accordance with various embodiments of these teachings;

FIG. 4 comprises a graph as configured in accordance with various embodiments of these teachings; and

FIG. 5 comprises a schematic representation as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, these various embodiments are suitable for use with a radiation treatment platform having a set of at least two selectable therapeutic energy settings and wherein the radiation treatment platform includes a therapeutic energy source that travels along an arcuate pathway about a given patient during a radiation treatment session (such as, for example, a volumetric modulated arc therapy radiation treatment session). By one approach, a control circuit can optimize therapeutic energy settings from amongst the set of at least two selectable therapeutic energy settings for different portions of the arcuate pathway to provide optimized therapeutic energy settings and then generate an optimized energy treatment plan as a function, at least in part, of the optimized therapeutic energy settings.

By one approach, optimizing the therapeutic energy settings can include optimizing a mix of photon-based and electron-based radiation treatment modalities. In particular, beam energy modes can be switched between photon and electron modes during the treatment session, for use in different arc portions.

By one approach, in lieu of the foregoing or in combination therewith, optimizing the therapeutic energy settings can comprise optimizing at least one of the therapeutic energy settings and a length for at least one of the different portions of the arcuate pathway as a function of a geometry-based analysis. These teachings will also accommodate optimizing the therapeutic energy settings by first conducting a dose optimization prior to optimizing the therapeutic energy settings.

By one approach, the aforementioned different portions of the arcuate pathway are each at least a pre-selected minimal length (such as, by one example, at least a fifteen degree portion of the arcuate pathway). By one approach, these teachings will accommodate automatically determining a length of at least one of the different portions of the arcuate pathway while optimizing the therapeutic energy settings.

By one approach, generating the optimized energy treatment plan as a function, at least in part, of the optimized therapeutic energy settings can comprise optimizing dose administration only subsequent to optimizing the therapeutic energy settings. These teachings will also accommodate, if desired, generating the optimized energy treatment plan as a function, at least in part, of the optimized therapeutic energy settings by conducting at least a portion of the optimization of the therapeutic energy settings while also optimizing dose.

So configured, these teachings will allow use of a combination of different energies within a single treatment field. That capability, in turn, can help facilitate a more precise and efficacious treatment. (It should be understood that this combination of different energies within a single treatment field will typically apply, at least in many application settings, to the optimization phase only. Many delivery systems are not configured to switch energies within a single field. Once optimized, however, one can split the single field that uses multiple energies into corresponding multiple fields where each resultant field has only a single energy and zero dose in sectors where other energies will apply.)

Volumetric modulated arc therapy is an approach that uses single or multiple arc fields. Since the gantry is rotating during the delivery of the field, the viewpoint from the radiation source to the target and healthy tissue changes during the arc. This means that a single selected energy might not be optimal for all parts of a given field. The present teachings will permit automatically selecting and/or optimizing the energies for individual sectors of an arc in a step before the final plan dose optimization and hence help to ensure a more efficacious treatment result.

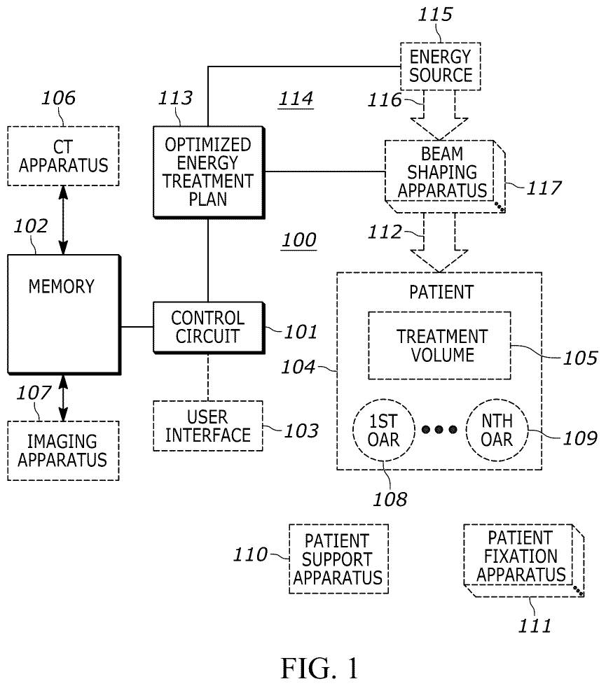

These and other benefits may become clearer upon making a thorough review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

It will be appreciated that the control circuit 101 may comprise a single integrated platform or may comprise a plurality of such circuits that work in cooperation with one another.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101). As with the control circuit 101, the memory 102 may comprise a singular structure or may comprise a plurality of memory platforms that collectively comprise the “memory” of this apparatus 100.

In addition to information such as optimization information for a particular patient and information regarding a particular radiation treatment platform as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless platforms, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan 113 (such as, for example, an optimized radiation treatment plan). This energy-based treatment plan 113 typically comprises specified values for each of a variety of treatment-platform parameters during each of a plurality of sequential exposure fields. In this case the energy-based treatment plan 113 is generated through an optimization process, examples of which are provided further herein.

By one approach the control circuit 101 can operably couple to an energy-based treatment platform 114 that is configured to deliver therapeutic energy 112 to a corresponding patient 104 having at least one treatment volume 105 and also one or more organs-at-risk (represented in FIG. 1 by a first through an Nth organ-at-risk 108 and 109) in accordance with the optimized energy-based treatment plan 113. These teachings are generally applicable for use with any of a wide variety of energy-based treatment platforms (which platforms are structural in general and comprised of a variety of apparatuses).

In a typical application setting the energy-based treatment platform 114 will include an energy source such as a radiation source 115 of ionizing radiation 116.

By one approach this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment platform 114 may also include one or more support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

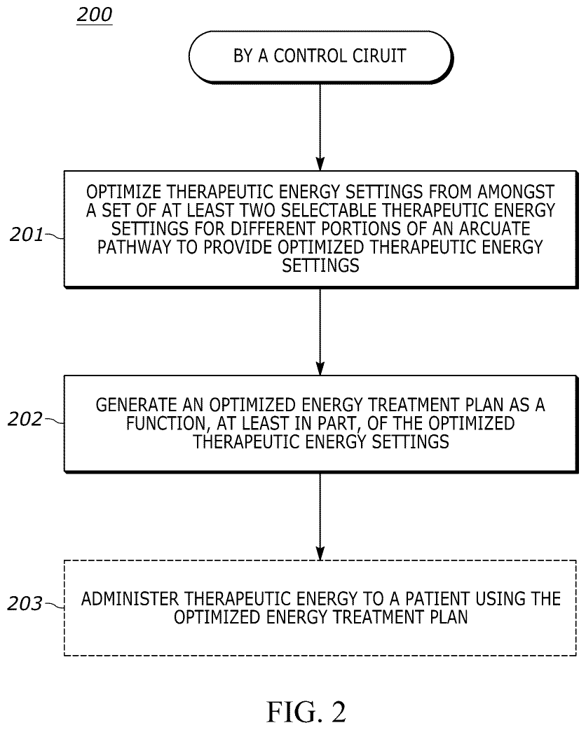

Referring now to FIG. 2, a process 200 that can be carried out, for example, in conjunction with the above-described application setting (and more particularly via the aforementioned control circuit 101) will be described. Generally speaking, this process 200 serves to facilitate generating an optimized radiation treatment plan to thereby facilitate treating a particular patient with therapeutic radiation using a particular radiation treatment platform per that optimized radiation treatment plan.

For the sake of an illustrative example, this description presumes the availability and use of a radiation treatment platform having a set of at least two selectable therapeutic energy settings and wherein the radiation treatment platform includes a therapeutic energy source (such as the above-described energy source 115) that travels along an arcuate pathway about a given patient during a radiation treatment session. Again for the sake of an illustrative example, the aforementioned radiation treatment session can comprise a volumetric modulated arc therapy radiation treatment session.

At block 201, this process 200 provides for the control circuit 101 optimizing therapeutic energy settings from amongst the aforementioned set of at least two selectable therapeutic energy settings for different portions of the aforementioned arcuate gantry pathway to provide optimized therapeutic energy settings. The energy settings can therefore be different at different portions of the arcuate gantry pathway, although such a result is not a mandatory result. Instead, the differences, when present, will be the result of the optimization process.

By one approach, the aforementioned different portions of the arcuate pathway are each at least a pre-selected minimal length (such as, for example, at least a ten degree, fifteen degree, or twenty degree portion of the arcuate pathway). Those different portions may all be intentionally of a same length, or the lengths may vary as desired (while still being at least that pre-selected minimal length). Imposing a minimal length for these different portions can help to minimize a number of energy changes that might require an undue amount of time to effect in real time as the corresponding treatment is implemented.

By one approach, optimizing the therapeutic energy settings can include automatically determining a length of at least one of the different portions, up to and including all of the different portions, of the arcuate pathway. In this case, and again, the determined length may be at least a pre-selected minimal length as described above. The number of different portions may, or may not, be also determined via the optimization process (while possibly also applying a maximum number of different portions that may be so identified).

By one approach, the aforementioned optimizing of the therapeutic energy settings can comprise optimizing at least one of or both of the available selectable therapeutic energy settings and a length for at least one or more (or all) of the different portions of the arcuate pathway. More particularly, the foregoing optimization can be processed as a function of a geometry-based analysis that takes into account, for example, platform geometries and/or patient geometries as desired. By one approach, the foregoing optimization can be processed as a function of dose-based analysis that takes into account, for example, existing optimized dose.

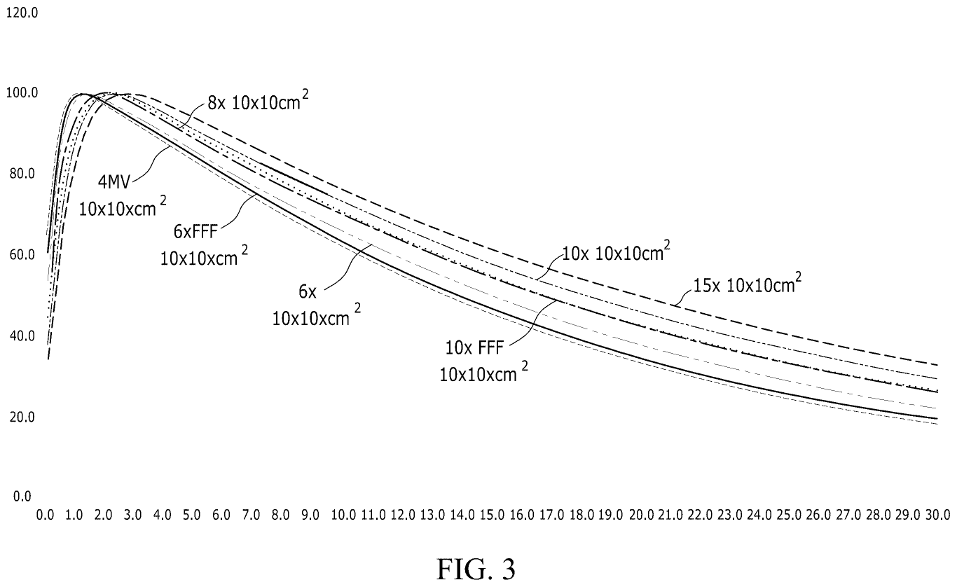

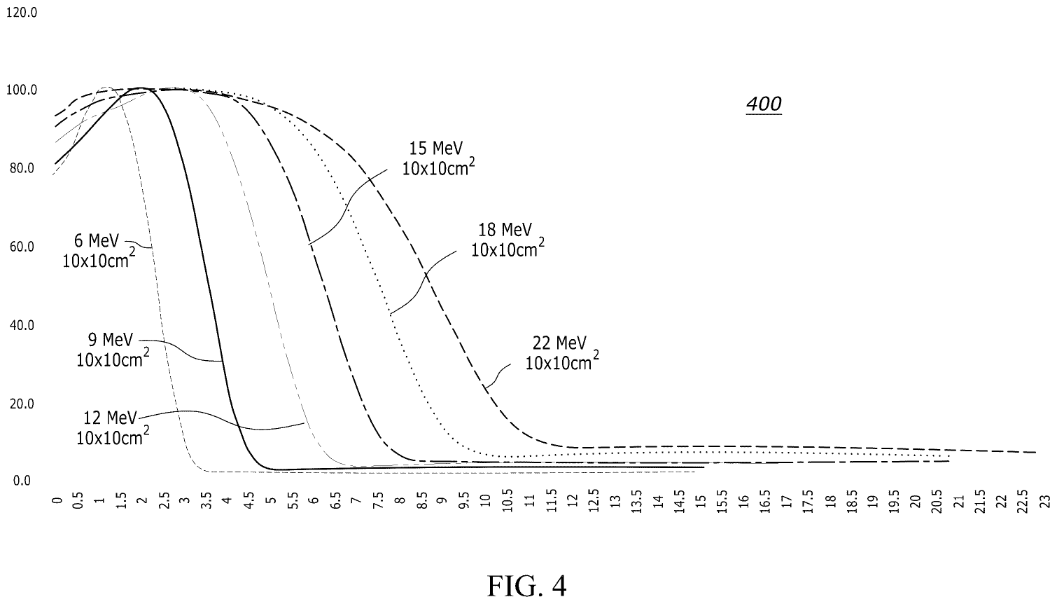

These teachings are highly flexible in practice and will accommodate, for example, optimizing the therapeutic energy settings by, at least in part, optimizing a mix of photon-based and electron-based radiation treatment modalities. Such an approach can leverage differences in resultant dose depths that can be achieved as between these two energy modalities. By way of generally illustrating such differences, FIG. 3 presents a graph 300 of photon depth dose curves, representing for example a relative absorbed amount of dose as a function of radiation travel length in a medium, for a given treatment platform while FIG. 4 presents a graph 400 of electron depth dose curves for the same treatment platform.

As another example in these regards, the optimizing of the therapeutic energy settings can include first conducting dose optimization prior to optimizing the therapeutic energy settings. Alternatively, if desired, these teachings will accommodate optimizing dose administration only subsequent to optimizing the therapeutic energy settings. And as yet another alternative, these teachings will accommodate optimizing dose administration prior to optimizing the therapeutic energy settings and then re-optimizing dose administration.

Upon concluding this pre-optimization round at block 201, a split arc setup can be created for current delivery units. Alternatively, when the energy treatment platform supports dynamic energy switching during a field, the arc can be retained in an original form, but additional beam-off control points without gantry rotation can be added for the platform to use for switching the energy levels.

At block 202, the control circuit 101 then subsequently generates an optimized energy treatment plan as a function, at least in part, of the aforementioned optimized therapeutic energy settings. If desired, generating the optimized energy treatment plan as a function, at least in part, of the optimized therapeutic energy settings can comprise conducting at least a portion of the optimization of the therapeutic energy settings while also optimizing dose. This can help adjusting the pre-optimized energy settings using the dose optimization cost function to find out what is a more optimal plan.

At optional block 203, this process 200 will further accommodate administering therapeutic energy to a patient using the aforementioned optimized treatment plan. Per these teachings, that plan may provide for using different energy levels for the administered energy at different portions of the energy source's pathway around the patient. As a specific example, these teachings can readily facilitate automatically determining different optimal energies for different parts of a volumetric modulated arc therapy field using available energy levels for the intended treatment platform.

Further details that comport with these teachings will now be presented. It will be understood that the specific details of these examples are intended to serve an illustrative purpose and are not intended to suggest any particular limitations with respect to these teachings.

These teachings will accommodate various approaches to optimizing optimal energy settings and arc sector lengths. By one such approach, a geometry-based analysis can be utilized. That analysis can be based on radiation paths that traverse healthy tissues as well as the patient's target volume for each angular direction within a sector represented by control points. (Control points refer to specific positions or angles at which a radiation beam is turned on or off during treatment. Control points typically serve to help shape the radiation beam and to optimize the dose distribution to target the tumor while sparing surrounding healthy tissues.)

In this analysis, useful observables can include three lengths, L1, L2 and L3, where each term could be a pair of two depth values, along a common path/trajectory described as follows: a first length, L1, from where the energy beam enters the patient's body to where the beam enters the target volume, a second length, L2, where the beam traverses the target volume, and a third length, L3, from where the beam exits the target to where the beam exits the patient's body. Depth dose curves, DDC(E), for each available energy mode option, E, can be accessed and used to select a particular optimum energy mode for each sector based on rules that favor selecting energy modes that have the most beneficial depth-dose-range for irradiating the target volume in the above-described second length and the least unwanted healthy tissue effects in the above-described first and third lengths. In other words, a geometry based quality metric can be formed as Q=Q(L1, L2, L3, DDC(E)). The quality metric gives higher values for more favorable energy depth dose curves and lower values for less favorable energy depth dose curves. The quality metric can be used in energy setting optimization for finding the best energy depth dose curve for each set of L1, L2, and L3 at a certain gantry angle. In one example, the function Q can be just a sum of depth dose curve areas at each depth range, L1, L2, L3, that simultaneously maximizes the area in the target volume part L2 and minimizes the areas in the healthy tissue parts L1 and L3.

The foregoing describes the analysis for one line at a particular angle but the geometry-based approach can use a three-dimensional representation of the structure volumes. In that case, a two-dimensional visibility map can be laid on the multi-leaf collimator plane, with some corresponding angle on the arcuate pathway thus formed, and a corresponding Q value can be calculated for each two-dimensional pixel value of interest in that map. For example, the visibility map can represent a target volume projection on the multi-leaf collimator plane at a certain angle. In this case the quality metric for that certain angle can be calculated by summing the Q values at each pixel in the set in that target projection.

By one approach, the quality metric can include a dose distribution as input. This can be useful since the geometrical approach usually does not include knowledge of the corresponding dose quality. For example, if the dose distribution is pre-optimized with some default energy settings, and using a similar or same dose quality function as what is being used in the final dose optimization, the quality metric includes more detailed spatial information about the underlaying structures, including the target.

The dose information can be used to weight different spatial locations in the geometrical information (for example, a sub-part in a target volume or a sub-part in healthy tissue) differently between each location in the Q function calculations. In one example, using the above example of calculating the curve area along some length L, the overlayed dose information along the length L can be used to change the value of the area calculation, either by reducing or increasing it according to the dose values along the line L. In typical examples, the dose along the line inside a target volume can change due to overlapping structure situations: the target volume can overlap with an OAR volume that was important to be avoided and the dose optimized to a low value in the overlap part, or the target volume can consist of different overlapping target volumes, each demanding different amounts of dose.

By one approach, the above considerations can comprise discretely examining the distances travelled by the energy beam in healthy tissue and in the target volume. When the distance through both is short, a low energy can be used. When the distance through healthy tissue is short and the distance through the target volume is relatively long, medium to high energy can be used. When the distance through healthy tissue is long and the distance through the patient volume is short to long (relatively speaking), a medium to high energy can be used. And when the distance through both the healthy tissue and the target volume is long, a relatively high energy level can be used.

By one approach, specific energy values can be fetched from a precalculated table of values. This approach can be relatively fast, but may not always fully accord with or properly observe clinical goals.

To avoid too many energy changes during the treatment session, minimum arc length requirements can be imposed as described above.

By one approach, these teachings will accommodate conducting dose-based optimization before the actual plan dose optimization using objectives/clinical goals at least for target coverage and healthy tissue sparing. Note that during this pre-optimization, certain energy mode needs to be selected (e.g. a most common energy mode) because the selected energy mode will affect achievable dose distributions. (Clinical goals are the treatment goals being prescribed by, for example, an attending oncologist. Examples of clinical goals include, but are not limited to, goals regarding the dose distributions to be achieved with respect to a target volume, one or more organs-at-risk (OAR) in the vicinity of the target volume, or other specified or unspecified normal tissues. By their very nature, clinical goals are typically defined prior the optimization and it is considered that the outcome of the dosimetric optimization or the selected energy mode does not necessarily affect the goals. Optimization objectives, on the other hand, will be understood to be objectives that are very much specifically designed to reflect and accommodate the technical details and specifications of a particular energy treatment platform, specific details regarding the patient's presentation, and/or other physical details pertaining to a particular application setting. Such details are generally viewed as being outside the expertise and knowledge base of the person who prescribes the radiation treatment in the first place (i.e., for example, a licensed oncologist). As a result, the person prescribing the radiation treatment ordinarily does not also create the optimization objectives.)

For example, a normal tissue objective can be used to find optimum energies and/or beginning and end points for the different arc sectors. Healthy tissues are not necessarily uniformly important and there can be a need to protect different organs/tissues with varying levels of protection. By employing a dose optimization engine at this point in the process, different dose-volume objectives and clinical goals can be taken more optimally into account. By one approach, during this optimization a discrete cost term can be created for use in the total cost function for controlling (or at least influencing) switching between different energies.

By yet another approach, these teachings will accommodate conducting energy mode optimization during the plan dose optimization: the energy modes (E) at all control points would be considered as additional degrees-of-freedom to be optimized simultaneously to the degrees-of-freedom (T) used in prior-art plan dose optimization (such as the leaf positions, jaw positions, and the amount of delivered radiation dose) by minimizing a cost function:

$$C ( T , E ) = {C}_{dos} ( T , E ) + {C}_{e} ( E )$$

where the first term C_{dos} corresponds the cost function used in prior art dosimetrical optimization (with the exception that the dosimetrical evaluation also takes into account E). The second term Cₑ is new and contains any contribution of the changing energy modes that is not directly affecting the dosimetrical quantities, such as a request to keep the energy mode constant at least a given angle interval (C_{e,arcl}(E)), or the desire to not have unnecessarily many energy changes which would increase the delivery time (C_{e,time}(E)):

$${C}_{e} ( E ) = {C}_{e , arcl} ( E ) + {C}_{e , time} ( E ) .$$

As an example the cost term for the increased treatment time due to energy changes can be expressed as

$${C}_{e , time} ( E ) = τ {\sum }_{i} ( 1 - {δ}_{{E}_{i} , {E}_{i + 1}} )$$

where δ_{E}_{ᵢ}_{,E}_{ᵢ₊₁} . . . is the Dirac's delta symbol getting value 1 if Eᵢ=Eᵢ₊₁ and 0 otherwise. Eᵢ is the energy mode associated to the control point index i. τ is the cost associated to the amount of time increase due to single energy mode switch and its purpose is to balance the importance of short delivery time with the dosimetrical plan quality.

As an example the penalty term associated to the desired length of arclets with constant energy can be presented as

$${C}_{e , arcl} ( E ) = α {\sum }_{i} θ ( {\sum }_{j ∈ [ i , i + k ]} ( 1 - {δ}_{{E}_{j} , {E}_{j + 1}} ) - 1 ) ,$$

where θ is Heaviside step function and the constant k has been chosen so that the desired minimum arclet between two energy mode changes is containing k control points. The penalty strength α is chosen so that the constraint is obtained with desired accuracy. Note that there are also prior art solutions for handling such requests as a true constraint (not allowing the optimizer to consider at all solutions violating the set minimum distance between two energy mode changes).

The optimization can be performed using any prior art optimization method that is capable to combine continuous and discreet degrees of freedom, such as Mixed Integer Programming, Simulated Annealing, Differential Evolution algorithms, ect.

It is also possible to treat the energy as a continuous degree-of-freedom (in order to use optimization methods that are not able to handle discreet degrees-of-freedom: In a first phase of the optimization process, the energy (Eopt) can change in a non-discrete way (i.e., over a fully or relatively unrestricted energy level range) but in a second phase the energies can be fixed for each sector by adjusting the fixed energy from the unrestricted value to a nearest energy setting (Efix) that is available on the energy treatment machine. If the optimizer determines, for example, a non-discrete value of Eopt<8.5 MV, that value can then be switched to 6 MV (the latter being, in this example, an energy level that is available while the former level is not). For an energy level of 8.5 MV≤Eopt<12.5 MV, the energy level may similarly be switched to an available energy level of 10 MV. As yet another illustrative example, a determined (but unavailable) value of 12.5 MV≤Eopt can be switched to an available energy level of 15 MV. Treating energy mode as a continuous variable requires that the dosimetrical evaluation of the current plan can be done using arbitrary energy. A reasonable (but not only possible) approach would be to evaluate the dose separately using the two nearest Efix (Efix1 and Efix2) and then interpolate the dose between these. So dose at certain arclet having constant energy is obtained as

$$D ( T , {E}_{opt} ) = \frac{{E}_{fix 2} - {E}_{opt}}{{E}_{fix 2} - {E}_{fix 2}} {D}_{{E}_{fix 1}} ( T ) + \frac{{E}_{opt} - {E}_{fix 1}}{{E}_{fix 2} - {E}_{fix 1}} {D}_{{E}_{fix 2}} ( T ) ,$$

where D_{E}_{_{fix1}} (T) and D_{E}_{_{fix2}} (T) are prior art dose calculation engines able to calculate dose for control point sequence T using either fixed energy Efix1 or Efix2. Note also that the proposed formula makes it possible to evaluate gradient of C_{dos}(T, E) (assumed to be a function of the dose) relative to Eopt.

In lieu of the foregoing, or possibly in combination therewith, the discrete energy levels can be reached by applying an increasingly stiff penalty term in the cost function that penalizes energies depending upon how distant the energy level is from a selected set of discrete energy levels. As an example, the following term could be added to Cₑ(E):

$${C}_{e , disc} = {\sum }_{{E}_{opt} ∈ E} {γ ( {E}_{opt} - {E}_{fix} )}^{2} ,$$

where the sum goes over all control points and the difference between the individual Eopt and the associated nearest Eext is penalized with a scalar γ that is increased over the course of the optimization.

It will be noted that these teachings will accommodate, in any of the aforementioned optimization approaches, prioritizing particular energies. This prioritization can comprise, for example, introducing a bias to the cost function. Such a bias can serve, for example, to prefer a lower energy option when two energy levels otherwise evaluate equally (either literally or within some predetermined range of equivalence). As an example, the following term could be added to Cₑ(E):

$${C}_{e , disc} = {\sum }_{{E}_{opt} ∈ E} β {E}_{opt} ,$$

where the sum goes over all control points and the difference between the individual Eopt and the coefficient β is used to define the strength of the desired to use smaller energies relative to the other terms in the cost function.

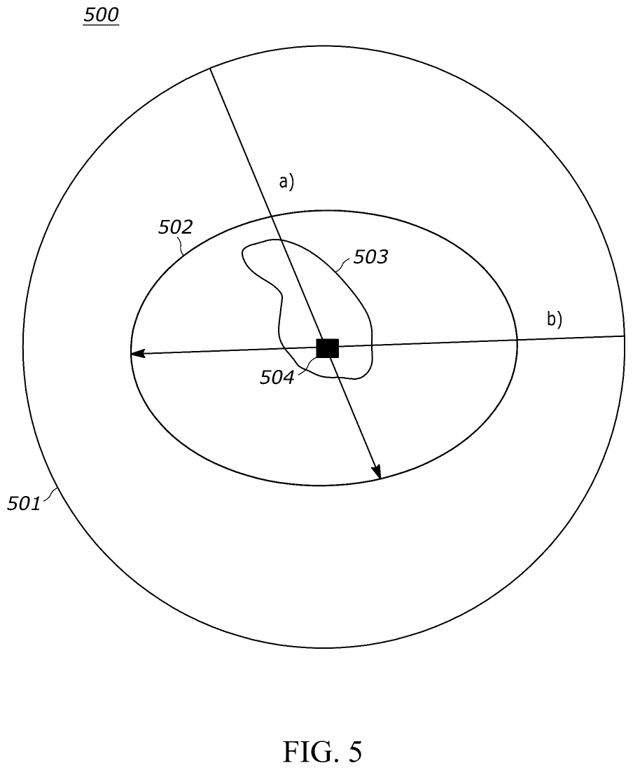

FIG. 5 presents an illustrative schematic example of a volumetric modulated arc therapy plan setup 500. In this example, the encompassing circle 501 represents the overall arc field. The ellipsoid 502 represents the body outline of the patient. The irregular shape 503 represents the target volume. And the black square 504 represents the isocenter of the arc field 501.

This illustration includes two rays denoted as “a” and “b.” Along these rays one can evaluate the effect of having specific photon depth dose curve characteristics for different locations along each ray. These locations along each ray can include, in particular, a non-targeted body part before the target volume 503, a location within the target volume 503, and a non-targeted body part that lies past the target volume 503 (all from the perspective of the beam's eye). (In the optional case of mixing photons and electrons, the evaluation of each ray could lead to selecting an electron depth dose characteristic considered to be a most beneficial one.

When considering ray “a,” the beam will reach the target volume 503 relatively shortly after entering the patient's body 502. This relative orientation and result can indicate that a lower photon energy can be more beneficial than a higher photon energy. In addition, a relatively large amount of healthy tissue remains in the path of this ray “a” after the beam leaves the target volume 503. This observation is a second indication that a lower energy may be more beneficial than a higher energy to avoid unnecessary increased doses in healthy tissue on the trailing side of the ray “a.”

Ray “b” presents a different scenario than ray “a.” Ray “b,” after entering the patient's body 502, must pass through a relatively longer portion of healthy untargeted tissue before reaching the target volume 503. In this case, a higher energy than was used for the beam associated with ray “a” may better help to reduce the dose in the healthy tissue in front of the target volume 503.

So configured, it will be appreciated that these teachings can provide for a better automated usage of different photon energies that may be available on modern multi-energy machines, or even automated usage of combined photon and electron energies for machines that can dynamically support those modalities. Automating the energy assignment for different parts on an arc field can help to reduce dose to healthy tissue while also maintaining desired coverage of the target volume. It will be appreciated that these teachings do not depend on the planners' skills. Therefore, more patients can potentially benefit from lowered dosing to healthy tissue.

Further aspects of the invention are provided by the subject matter of the following clauses (where it will be understood that any of these clauses can be combined with any one of more of the other clauses as appropriate):

Clause 1. A method for use with a radiation treatment platform having a set of at least two selectable therapeutic energy settings and wherein the radiation treatment platform includes a therapeutic energy source that travels along an arcuate pathway about a given patient during a radiation treatment session, the method comprising: by a control circuit: optimizing therapeutic energy settings from amongst the set of at least two selectable therapeutic energy settings for different portions of the arcuate pathway to provide optimized therapeutic energy settings; generating an optimized energy treatment plan as a function, at least in part, of the optimized therapeutic energy settings.

Clause 2. The method of clause 1 wherein the radiation treatment session comprises a volumetric modulated arc therapy radiation treatment session.

Clause 3. The method of clause 1 wherein the different portions of the arcuate pathway are each at least a pre-selected minimal length.

Clause 4. The method of clause 3 wherein the pre-selected minimal length is at least a fifteen degree portion of the arcuate pathway.

Clause 5. The method of clause 3 wherein optimizing the therapeutic energy settings includes automatically determining a length of at least one of the different portions of the arcuate pathway.

Clause 6. The method of clause 1 wherein generating an optimized energy treatment plan as a function, at least in part, of the optimized therapeutic energy settings comprises optimizing dose administration only subsequent to optimizing the therapeutic energy settings.

Clause 7. The method of clause 1 wherein optimizing the therapeutic energy settings includes optimizing a mix of photon-based and electron-based radiation treatment modalities.

Clause 8. The method of clause 1 wherein optimizing the therapeutic energy settings comprises optimizing at least one of: the therapeutic energy settings; and a length for at least one of the different portions of the arcuate pathway; as a function of a geometry-based analysis.

Clause 9. The method of clause 1 wherein optimizing the therapeutic energy settings includes first conducting a dose optimization prior to optimizing the therapeutic energy settings.

Clause 10. The method of clause 1 wherein generating the optimized energy treatment plan as a function, at least in part, of the optimized therapeutic energy settings comprises conducting at least a portion of the optimization of the therapeutic energy settings while also optimizing dose.

Clause 11. An apparatus for use with a radiation treatment platform having a set of at least two selectable therapeutic energy settings and wherein the radiation treatment platform includes a therapeutic energy source that travels along an arcuate pathway about a given patient during a radiation treatment session, the apparatus comprising: a control circuit configured to: optimize therapeutic energy settings from amongst the set of at least two selectable therapeutic energy settings for different portions of the arcuate pathway to provide optimized therapeutic energy settings; generate an optimized energy treatment plan as a function, at least in part, of the optimized therapeutic energy settings.

Clause 12. The apparatus of clause 11 wherein the radiation treatment session comprises a volumetric modulated arc therapy radiation treatment session.

Clause 13. The apparatus of clause 11 wherein the different portions of the arcuate pathway are each at least a pre-selected minimal length.

Clause 14. The apparatus of clause 13 wherein the pre-selected minimal length is at least a fifteen degree portion of the arcuate pathway.

Clause 15. The apparatus of clause 13 wherein the control circuit is configured to optimize the therapeutic energy settings by, at least in part, automatically determining a length of at least one of the different portions of the arcuate pathway.

Clause 16. The apparatus of clause 11 wherein the control circuit is configured to generate an optimized energy treatment plan as a function, at least in part, of the optimized therapeutic energy settings by optimizing dose administration only subsequent to optimizing the therapeutic energy settings.

Clause 17. The apparatus of clause 11 wherein the control circuit is configured to optimize the therapeutic energy settings by, at least in part, optimizing a mix of photon-based and electron-based radiation treatment modalities.

Clause 18. The apparatus of clause 11 wherein the control circuit is configured to optimize the therapeutic energy settings by, at least in part, optimizing at least one of: the therapeutic energy settings; and a length for at least one of the different portions of the arcuate pathway; as a function of a geometry-based analysis.

Clause 19. The apparatus of clause 11 wherein the control circuit is configured to optimize the therapeutic energy settings by, at least in part, first conducting a dose optimization prior to optimizing the therapeutic energy settings.

Clause 20. The apparatus of clause 11 wherein the control circuit is configured to generate the optimized energy treatment plan as a function, at least in part, of the optimized therapeutic energy settings by conducting at least a portion of the optimization of the therapeutic energy settings while also optimizing dose.

Those skilled in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

1. A method for use with a radiation treatment platform having a set of at least two selectable therapeutic energy settings and wherein the radiation treatment platform includes a therapeutic energy source that travels along an arcuate pathway about a given patient during a radiation treatment session, the method comprising:
by a control circuit:
optimizing therapeutic energy settings from amongst the set of at least two selectable therapeutic energy settings for different portions of the arcuate pathway to provide optimized therapeutic energy settings;
generating an optimized energy treatment plan as a function, at least in part, of the optimized therapeutic energy settings.

2. The method of claim 1 wherein the radiation treatment session comprises a volumetric modulated arc therapy radiation treatment session.

3. The method of claim 1 wherein the different portions of the arcuate pathway are each at least a pre-selected minimal length.

4. The method of claim 3 wherein the pre-selected minimal length is at least a fifteen degree portion of the arcuate pathway.

5. The method of claim 3 wherein optimizing the therapeutic energy settings includes automatically determining a length of at least one of the different portions of the arcuate pathway.

6. The method of claim 1 wherein generating an optimized energy treatment plan as a function, at least in part, of the optimized therapeutic energy settings comprises optimizing dose administration only subsequent to optimizing the therapeutic energy settings.

7. The method of claim 1 wherein optimizing the therapeutic energy settings includes optimizing a mix of photon-based and electron-based radiation treatment modalities.

8. The method of claim 1 wherein optimizing the therapeutic energy settings comprises optimizing at least one of:
the therapeutic energy settings; and
a length for at least one of the different portions of the arcuate pathway;
as a function of a geometry-based analysis.

9. The method of claim 1 wherein optimizing the therapeutic energy settings includes first conducting a dose optimization prior to optimizing the therapeutic energy settings.

10. The method of claim 1 wherein generating the optimized energy treatment plan as a function, at least in part, of the optimized therapeutic energy settings comprises conducting at least a portion of the optimization of the therapeutic energy settings while also optimizing dose.

11. An apparatus for use with a radiation treatment platform having a set of at least two selectable therapeutic energy settings and wherein the radiation treatment platform includes a therapeutic energy source that travels along an arcuate pathway about a given patient during a radiation treatment session, the apparatus comprising:
a control circuit configured to:
optimize therapeutic energy settings from amongst the set of at least two selectable therapeutic energy settings for different portions of the arcuate pathway to provide optimized therapeutic energy settings;
generate an optimized energy treatment plan as a function, at least in part, of the optimized therapeutic energy settings.

12. The apparatus of claim 11 wherein the radiation treatment session comprises a volumetric modulated arc therapy radiation treatment session.

13. The apparatus of claim 11 wherein the different portions of the arcuate pathway are each at least a pre-selected minimal length.

14. The apparatus of claim 13 wherein the pre-selected minimal length is at least a fifteen degree portion of the arcuate pathway.

15. The apparatus of claim 13 wherein the control circuit is configured to optimize the therapeutic energy settings by, at least in part, automatically determining a length of at least one of the different portions of the arcuate pathway.

16. The apparatus of claim 11 wherein the control circuit is configured to generate an optimized energy treatment plan as a function, at least in part, of the optimized therapeutic energy settings by optimizing dose administration only subsequent to optimizing the therapeutic energy settings.

17. The apparatus of claim 11 wherein the control circuit is configured to optimize the therapeutic energy settings by, at least in part, optimizing a mix of photon-based and electron-based radiation treatment modalities.

18. The apparatus of claim 11 wherein the control circuit is configured to optimize the therapeutic energy settings by, at least in part, optimizing at least one of:
the therapeutic energy settings; and
a length for at least one of the different portions of the arcuate pathway;
as a function of a geometry-based analysis.

19. The apparatus of claim 11 wherein the control circuit is configured to optimize the therapeutic energy settings by, at least in part, first conducting a dose optimization prior to optimizing the therapeutic energy settings.

20. The apparatus of claim 11 wherein the control circuit is configured to generate the optimized energy treatment plan as a function, at least in part, of the optimized therapeutic energy settings by conducting at least a portion of the optimization of the therapeutic energy settings while also optimizing dose.
